(19) United States
(12) Patent Application Publication
(10) Pub. No.: US 2019/0223834 A1
(43) Pub. Date: Jul. 25, 2019

(54) ULTRASONIC PROBE HOLDER

(71) Applicant: Konica Minolta, Inc., Tokyo (JP)

(72) Inventors: Masayuki NUKAYA, Sagamihara-shi (JP); Takahiko Shiraishi, Tokyo (JP); Shinya Noguchi, Tokyo (JP); Tatsushi Chihara, Tokyo (JP)

(21) Appl. No.: 16/241,459

(22) Filed: Jan. 7, 2019

(30) Foreign Application Priority Data
Jan. 23, 2018 (JP) 2018-008730

(51) Int. Cl. A61B 8/00

(52) U.S. Cl. CPC A61B 8/4209; A61B 8/4444

(57) ABSTRACT

An ultrasonic probe holder which holds and houses an ultrasonic probe including a head including an output surface which outputs ultrasonic waves and a grip extending from the head includes: a bottom that includes a first opening having a size such that the head and the grip of the ultrasonic probe do not pass therethrough; and a tube that extends from the bottom and includes a second opening having a size such that the head and the grip of the ultrasonic probe pass therethrough at an extended end and a slit connected to the second opening and the first opening on the bottom.

The entire disclosure of Japanese patent Application No. 2018-008730, filed on Jan. 23, 2018, is incorporated herein by reference in its entirety.

### BACKGROUND

### Technological Field

The present invention relates to an ultrasonic probe holder which holds and houses an ultrasonic probe.

### Description of the Related Art

An ultrasonic diagnostic device which transmits and receives ultrasonic waves to and from a subject such as a living body by an ultrasonic probe, generates ultrasonic image data on the basis of a signal obtained from the received ultrasonic waves, and displays an ultrasonic image based on the same on an image display device is conventionally known. Ultrasonic diagnosis using such a device is such that a state of the subject such as a heartbeat or movement of a fetus may be obtained in real time by simple operation of putting the ultrasonic probe on a body surface of the subject, and this is noninvasive and safe, so that this may be repeatedly performed.

JP 07-327996 A discloses a holder of an ultrasonic probe improved so as to prevent breakage and scratching of an acoustic lens surface of the ultrasonic probe. In JP 07-327996 A, a bottom surface of the holder is formed to have a substantially V-shaped cross-section so that the acoustic lens does not come into contact with the bottom surface of the holder to be damaged or the like.

However, in the holder of JP 07-327996 A, the ultrasonic probe is housed and held such that the acoustic lens surface faces downward (face the bottom surface of the holder). Therefore, there is a problem that a gel applied to the acoustic lens surface drops on the bottom surface when the ultrasonic probe is used.

### SUMMARY

Therefore, an object of the present invention is to provide an ultrasonic probe holder which houses the ultrasonic probe so as to protect an output surface thereof which outputs ultrasonic waves when the ultrasonic probe is not used, and holds the ultrasonic probe such that a gel does not drop when the ultrasonic probe is used.

To achieve the abovementioned object, according to an aspect of the present invention, an ultrasonic probe holder which holds and houses an ultrasonic probe including a head including an output surface which outputs ultrasonic waves and a grip extending from the head, reflecting one aspect of the present invention comprises: a bottom that includes a first opening having a size such that the head and the grip of the ultrasonic probe do not pass therethrough; and a tube that extends from the bottom and includes a second opening having a size such that the head and the grip of the ultrasonic probe pass therethrough at an extended end and a slit connected to the second opening and the first opening on the bottom.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a perspective view illustrating an example of an ultrasonic probe holder according to an embodiment of the present invention;

FIG. 2 is a view of the ultrasonic probe holder as seen from the back;

FIG. 3 is a perspective view of the ultrasonic probe holder, a holder support member, a screw, and a screw receiving plate;

FIG. 4 is a perspective view of the ultrasonic probe holder and an ultrasonic probe;

FIG. 5 is a view illustrating a state when the ultrasonic probe is held by the ultrasonic probe holder;

FIG. 6A is a view illustrating a state in which the ultrasonic probe is housed in the ultrasonic probe holder;

FIG. 6B is a view illustrating a state in which the ultrasonic probe is housed in the ultrasonic probe holder;

FIG. 7 is a perspective view of the ultrasonic probe holder, the holder support member, and the screw;

FIG. 8 is a view of the ultrasonic probe holder as seen from above;

FIG. 9 is a cross-sectional view as seen in arrow AA direction of FIG. 8;

FIG. 10 is a cross-sectional view as seen in arrow BB direction of FIG. 8;

FIG. 11 is a view for illustrating a housed state of the ultrasonic probe;

FIG. 12 is a view illustrating the ultrasonic probe holder and the holder support member when attaching the ultrasonic probe holder to an ultrasonic diagnostic device; and

FIG. 13 is a view illustrating the ultrasonic probe holder and the holder support member when housing the ultrasonic probe holder.

### DETAILED DESCRIPTION OF EMBODIMENTS

Hereinafter, one or more embodiments of the present invention will be described with reference to the drawings. However, the scope of the invention is not limited to the disclosed embodiments.

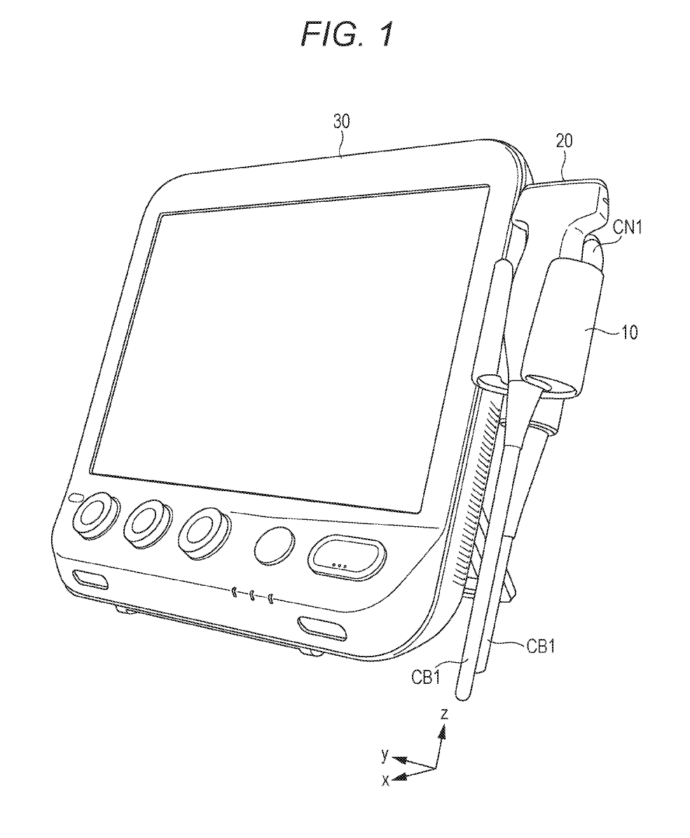

FIG. 1 is a perspective view illustrating an ultrasonic probe holder 10 according to the embodiment of the present invention. In FIG. 1, an ultrasonic probe 20 and an ultrasonic diagnostic device 30 are also illustrated. Hereinafter, an orthogonal coordinate system of x, y, and z axes illustrated in FIG. 1 is set for the ultrasonic probe holder 10 and the ultrasonic diagnostic device 30. In FIG. 1, the front or a front surface of the ultrasonic probe holder 10 and the ultrasonic diagnostic device 30 are in a +x axis direction, and the back or a back surface of the ultrasonic probe holder 10 and the ultrasonic diagnostic device 30 are in a −x axis direction. Also, an upper side of the ultrasonic probe holder 10 and the ultrasonic diagnostic device 30 is in a +z axis direction, and a lower side of the ultrasonic probe holder 10 and the ultrasonic diagnostic device 30 is in a −z axis direction.

The ultrasonic probe holder 10 is attached so as to be lateral to the ultrasonic diagnostic device 30. In FIG. 1, the ultrasonic probe holder 10 is attached to the ultrasonic diagnostic device 30 so as to be located on a right side of the ultrasonic diagnostic device 30.

The ultrasonic probe holder 10 is detachable from the ultrasonic diagnostic device 30. For example, the ultrasonic probe holder 10 is detached from the ultrasonic diagnostic device 30 when the ultrasonic diagnostic device 30 is housed in a housing case, a housing bag or the like to be carried. When the ultrasonic probe holder 10 is detached, the ultrasonic diagnostic device 30 has a substantially rectangular parallelepiped shape without a protruding portion by the ultrasonic probe holder 10. As a result, the ultrasonic diagnostic device 30 is easily housed in the housing case, the housing bag or the like without being caught thereby.

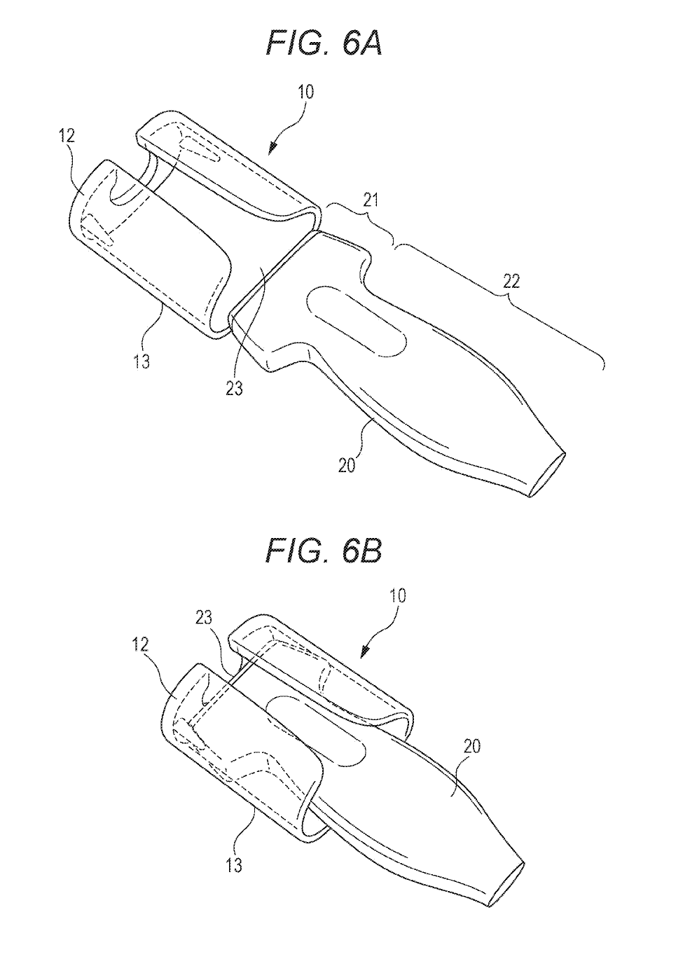

The ultrasonic probe holder 10 holds and houses the ultrasonic probe 20. For example, as illustrated in FIG. 1, the ultrasonic probe holder 10 attached to the ultrasonic diagnostic device 30 holds the ultrasonic probe 20 with a head of the ultrasonic probe 20 facing upward. Also, for example, the ultrasonic probe holder 10 detached from the ultrasonic diagnostic device 30 houses the ultrasonic probe 20 such that the head of the ultrasonic probe 20 faces a bottom of the ultrasonic probe holder 10 (refer to FIG. 6B).

The ultrasonic probe holder 10 holds the ultrasonic probe 20 such that the head of the ultrasonic probe 20 faces upward as illustrated in FIG. 1, thereby suppressing a gel applied to the head of the ultrasonic probe 20 from dropping.

On the other hand, the ultrasonic probe holder 10 houses the ultrasonic probe 20 such that the head of the ultrasonic probe 20 faces the bottom of the ultrasonic probe holder 10 (refer to FIG. 6B), thereby protecting the head of the ultrasonic probe 20. For example, when the ultrasonic probe 20 is housed in the housing case, the housing bag or the like together with or separately from the ultrasonic diagnostic device 30, the ultrasonic probe holder 10 protects the head of the ultrasonic probe 20 so as not to be brought into contact with the ultrasonic diagnostic device 30, the housing case, the housing bag or the like to be broken.

A cable CB1 extends from the ultrasonic probe 20. An end of the cable CB1 extending from the ultrasonic probe 20 is a connector CN1 connected to the ultrasonic diagnostic device 30 on a side surface of the ultrasonic diagnostic device 30. That is, the ultrasonic probe 20 is detachable from the ultrasonic diagnostic device 30. Note that, in FIG. 1, a part of the cable CB1 between the ultrasonic probe 20 and the connector CN1 is not illustrated.

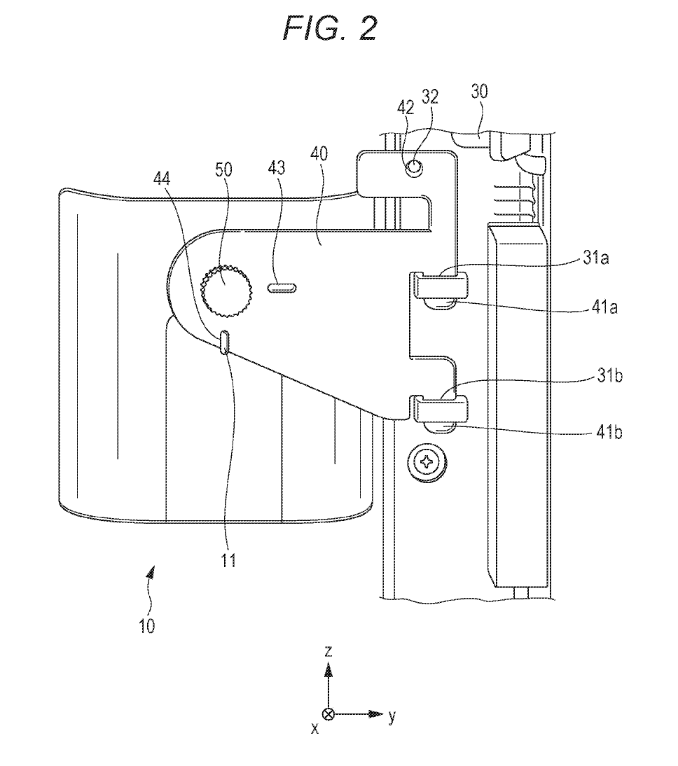

FIG. 2 is a view of the ultrasonic probe holder 10 as seen from the back. FIG. 2 illustrates a part of the back surface of the ultrasonic diagnostic device 30, a holder support member 40, and a screw 50. In FIG. 2, the same reference numerals are assigned to the same components as those in FIG. 1.

The ultrasonic diagnostic device 30 includes holes 31a and 31b penetrating in the z axis direction on the back surface thereof. Also, the ultrasonic diagnostic device 30 includes a protrusion 32 on the back surface thereof.

The holder support member 40 is a plate-shaped member and includes protrusions 41a and 41b and a hole 42. The protrusions 41a and 41b pass through the holes 31a and 31b provided on the back surface of the ultrasonic diagnostic device 30, respectively, and the hole 42 is fitted to the protrusion 32 provided on the back surface of the ultrasonic diagnostic device 30. The holder support member 40 is fixed to the back surface of the ultrasonic diagnostic device 30 by the protrusions 41a and 41b passing through the holes 31a and 31b provided on the back surface of the ultrasonic diagnostic device 30, and the hole 42 fitted to the protrusion 32 provided on the back surface of the ultrasonic diagnostic device 30. Also, the holder support member 40 is detachable from the back surface of the ultrasonic diagnostic device 30.

When the holder support member 40 is fixed to the back surface of the ultrasonic diagnostic device 30, a part thereof protrudes from the side surface of the ultrasonic diagnostic device 30. In an example in FIG. 2, a part of the holder support member 40 protrudes in a −y axis direction from the side surface of the ultrasonic diagnostic device 30.

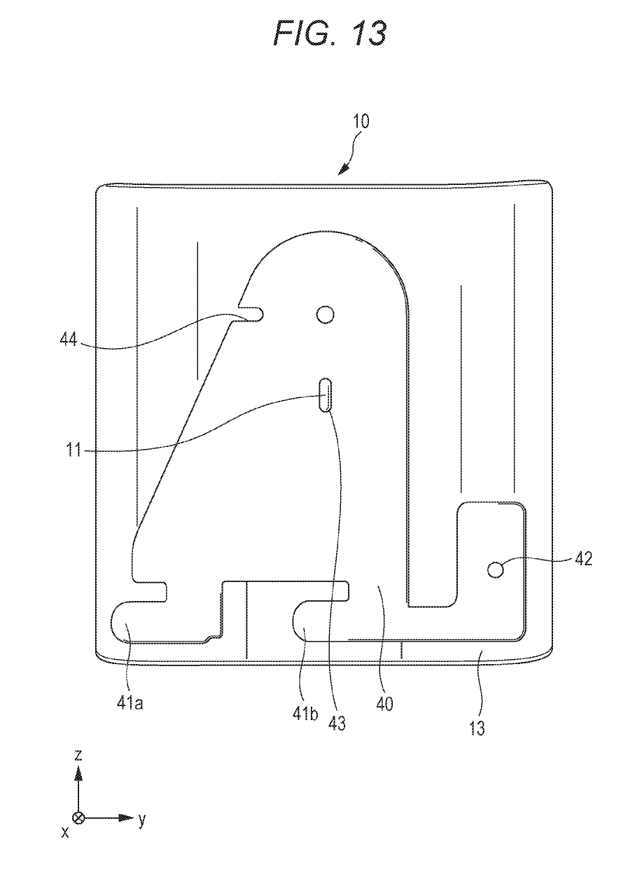

The holder support member 40 also includes a hole 43 and a cutout 44 (refer to FIG. 13). The hole 43 and the cutout 44 are adapted to be fitted to the protrusion 11 provided on the back surface of the ultrasonic probe holder 10. In the example in FIG. 2, the cutout 44 is fitted to the protrusion 11. A function of the hole 43 and the cutout 44 is to be described later.

The ultrasonic probe holder 10 is fixed to the holder support member 40 by the screw 50. As described above, since the holder support member 40 protrudes from the side surface of the ultrasonic diagnostic device 30, when the ultrasonic probe holder 10 is fixed to the holder support member 40 by the screw 50, this is located lateral to the ultrasonic diagnostic device 30.

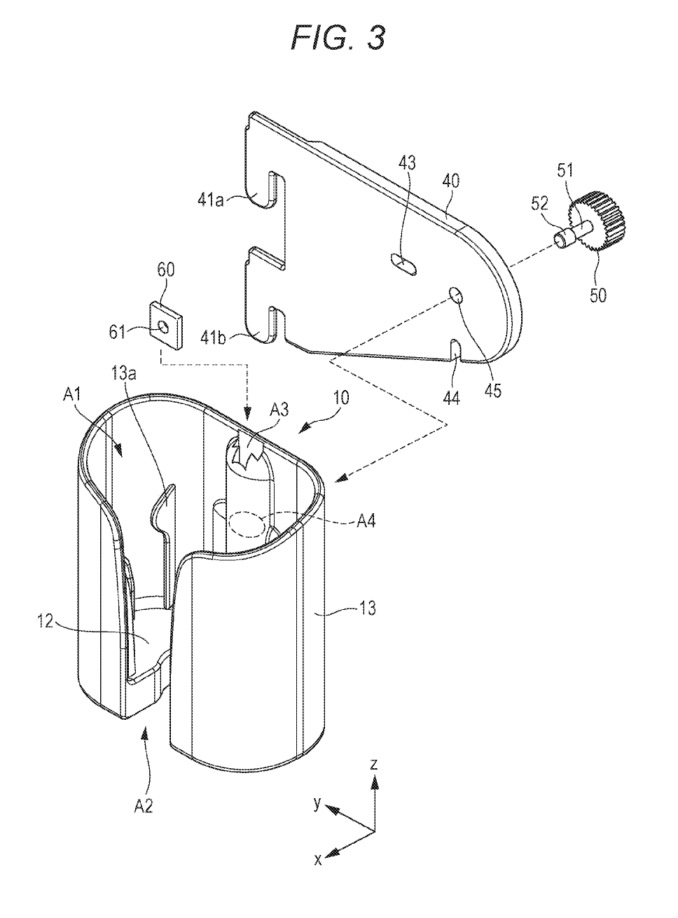

FIG. 3 is a perspective view of the ultrasonic probe holder 10, the holder support member 40, the screw 50, and a screw receiving plate 60. In FIG. 3, the same reference numerals are assigned to the same components as those in FIG. 2. Note that, in FIG. 3, a portion around the hole 42 of the holder support member 40 illustrated in FIG. 2 is not illustrated.

As illustrated in FIG. 3, the ultrasonic probe holder 10 includes a bottom 12 and a tube 13.

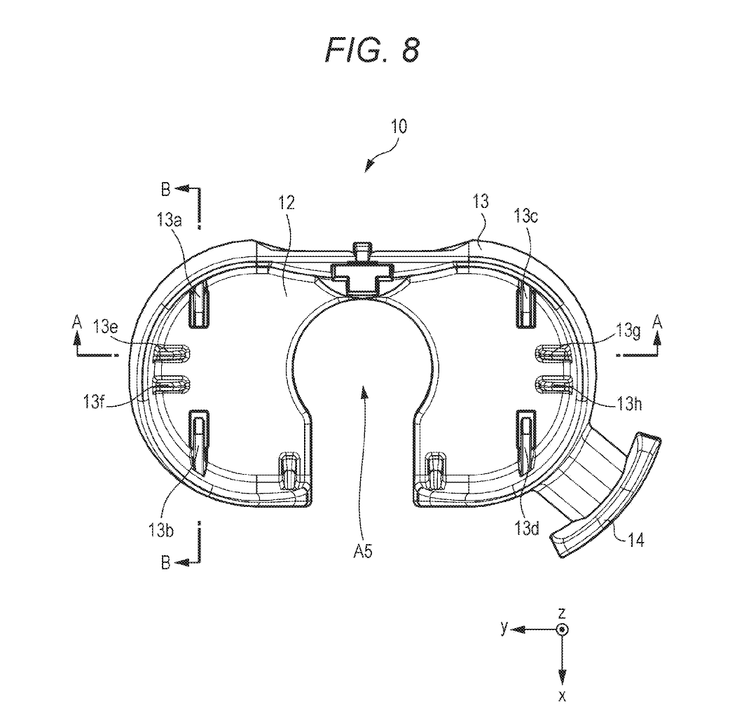

The bottom 12 in a plate shape has a substantially oval shape (refer to FIG. 8). The tube 13 has a tubular shape and extends from an outer periphery of the bottom 12 in the +z axis direction.

The tube 13 includes an opening A1 having a substantially oval shape at an end extended in the +z axis direction. Also, the tube 13 includes a slit A2 extending in the z axis direction on a front surface thereof. The slit A2 is connected to the opening A1.

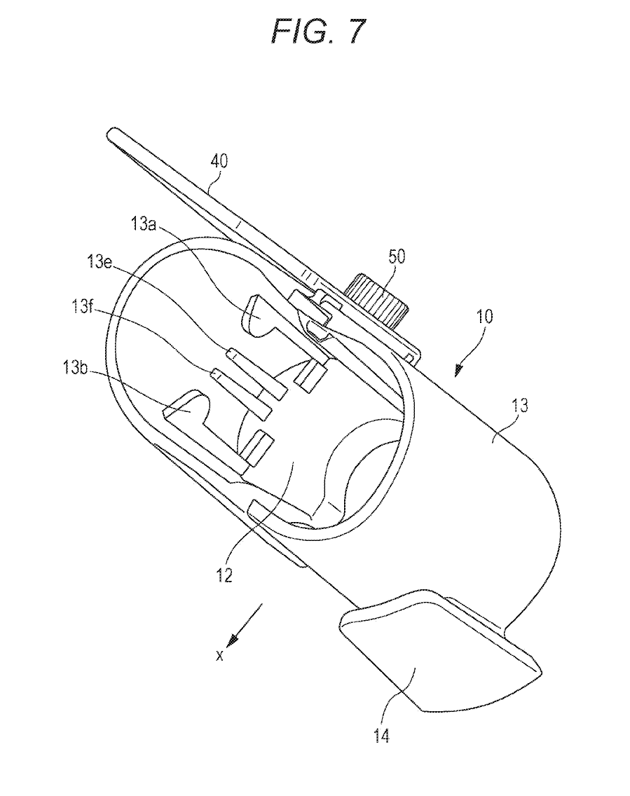

Also, the tube 13 includes a hole A3 extending in the −z axis direction from the opening A1 on an inner side on a back side thereof. Also, the tube 13 includes a protrusion 13a on the inner side thereof. Note that, in addition to the protrusion 13a, a plurality of protrusions is provided on the inner side of the tube 13 (refer to FIGS. 7 and 8). A function of the protrusion provided on the inner side of the tube 13 is to be described later.

The screw receiving plate 60 includes a hole 61 into which a screw portion 52 of the screw 50 screws. The screw receiving plate 60 is inserted into the hole A3 of the tube 13. The screw receiving plate 60 is inserted into the hole A3 to reach dotted line A4 illustrated in FIG. 3.

The screw 50 includes a cylindrical portion 51 and the screw portion 52 having a diameter larger than that of the cylindrical portion 51. The holder support member 40 includes a hole 45 having a diameter larger than that of the cylindrical portion 51 of the screw 50 and smaller than that of the screw portion 52 of the screw 50. Although the screw 50 is separated from the holder support member 40 in FIG. 3, the cylindrical portion 51 passes through the hole 45 of the holder support member 40. That is, the screw 50 is rotatably connected to the holder support member 40 so as not to separate from the holder support member 40. As a result, it is possible to prevent the screw 50 from being lost.

As described above, the screw receiving plate 60 into which the screw 50 screws is inserted into the hole A3 of the ultrasonic probe holder 10 to reach dotted line A4 illustrated in FIG. 3. The ultrasonic probe holder 10 may be fixed to the holder support member 40 by screwing the screw 50 into the hole 61 of the screw receiving plate 60 inserted into the hole A3 of the ultrasonic probe holder 10. Also, by separating the screw 50 from the screw receiving plate 60, the ultrasonic probe holder 10 may be detached from the holder support member 40. That is, the ultrasonic probe holder 10 is attachable to and detachable from the holder support member 40.

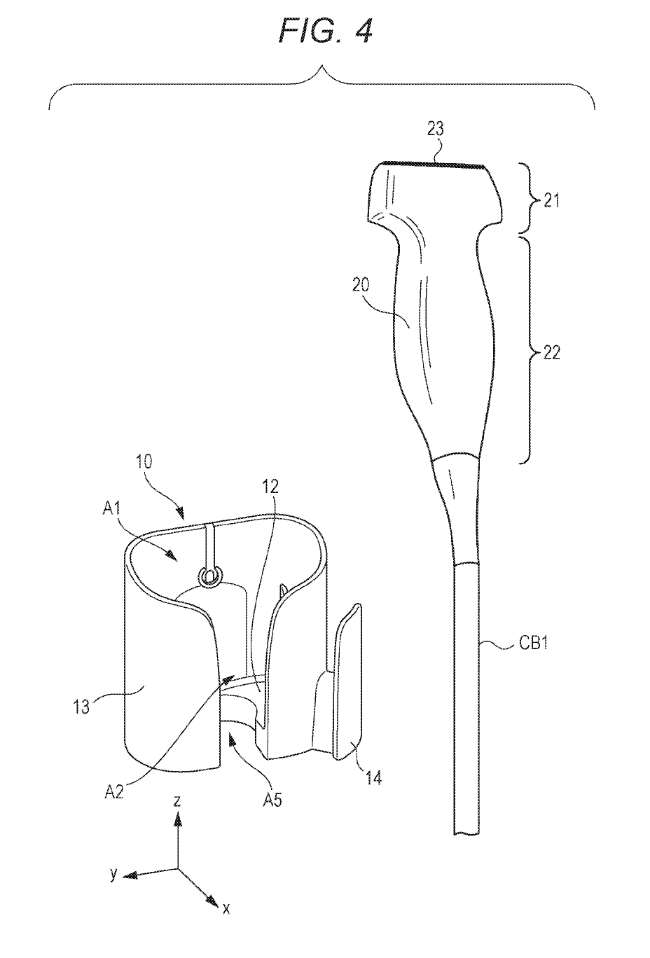

FIG. 4 is a perspective view of the ultrasonic probe holder 10 and the ultrasonic probe 20. In FIG. 4, the same reference numerals are given to the same components as those in FIGS. 1 to 3.

The ultrasonic probe 20 is divided into a head 21 and a grip 22. The head 21 includes an output surface 23 which outputs ultrasonic waves at an end on a side opposite to the grip 22. The cable CB1 extends from an end on a side opposite to the head 21 of the grip 22.

The bottom 12 of the ultrasonic probe holder 10 includes a substantially circular opening A5 at a substantially central portion thereof (refer to FIG. 8). The opening A5 of the bottom 12 is connected to the slit A2 provided on the front surface of the tube 13.

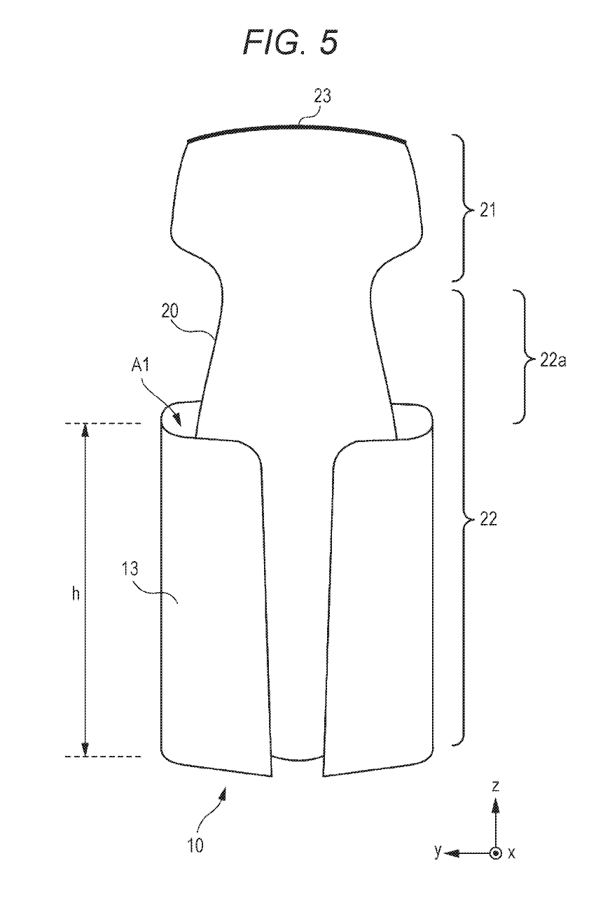

The opening A1 of the tube 13 has a size such that the grip 22 of the ultrasonic probe 20 may pass therethrough. Also, the tube 13 includes the slit A2 and the bottom 12 includes the opening A5 connected to the slit A2. Therefore, by allowing the cable CB1 to pass through the slit A2 of the tube 13 and pass through the opening A5 of the bottom 12, the ultrasonic probe 20 may be inserted into the tube 13 the grip 22 first (refer to FIGS. 1 and 5). Note that the slit A2 has at least a width through which the cable CB1 may pass.

Also, the opening A1 of the tube 13 has a size such that the head 21 of the ultrasonic probe 20 may pass therethrough. Therefore, the ultrasonic probe 20 may also be inserted into the tube 13 the head 21 first (refer to FIG. 6B).

The opening A5 of the bottom 12 has such a size that the head 21 and the grip 22 cannot pass therethrough. Therefore, in a case where the ultrasonic probe 20 is inserted into the tube 13 the grip 22 first, the grip 22 is supported by the bottom 12. That is, the ultrasonic probe 20 is held by the ultrasonic probe holder 10 in a case where this is inserted into the tube 13 the grip 22 first. Note that, as is described later, in a case where the ultrasonic probe 20 is inserted into the tube 13 the head 21 first, this is supported by the protrusion provided on the inner side of the tube 13.

The ultrasonic probe holder 10 includes a cable receiver 14 on an outer peripheral surface of the tube 13. The cable CB1 extending from the ultrasonic probe 20 is hung on or wound around the cable receiver 14.

For example, when the ultrasonic probe 20 is held by the ultrasonic probe holder 10 as illustrated in FIG. 1, the cable CB1 may be hung on the cable receiver 14. As a result, it is possible to suppress the cable CB1 from hanging down and coming into contact with a floor or the like. Also, for example, when the ultrasonic probe 20 is housed in the ultrasonic probe holder 10 (refer to FIG. 6B), the cable CB1 is wound around the cable receiver 14. This makes it easy to house the ultrasonic probe 20 housed in the ultrasonic probe holder 10 in the housing case, the housing bag or the like. Note that, in FIGS. 1 to 3, the cable receiver 14 is not illustrated.

FIG. 5 is a view illustrating a state when the ultrasonic probe 20 is held by the ultrasonic probe holder 10. In FIG. 5, the same reference numerals are assigned to the same components as those in FIGS. 1 to 4. Note that, in FIG. 5, the shape of the ultrasonic probe holder 10 is illustrated in a simplified manner. Also, in FIG. 5, the cable receiver 14 and the cable CB1 extending from the grip 22 of the ultrasonic probe 20 illustrated in FIG. 4 are not illustrated.

As illustrated in FIG. 5, a height h of the tube 13 of the ultrasonic probe holder 10 is smaller than a length of the grip 22 in a longitudinal direction (z axis direction). That is, the tube 13 has a height such that a part of the grip 22 gets out of the tube 13 when the ultrasonic probe 20 is inserted into the tube 13 such that the head 21 thereof faces upward (in other words, when the grip 22 is inserted from the opening A1 toward the bottom 12). For example, a part 22a of the grip 22 illustrated in FIG. 5 comes out of the tube 13.

As a result, a user may easily take the ultrasonic probe 20 held by the ultrasonic probe holder 10 from the ultrasonic probe holder 10. For example, the user may grasp the part 22a of the ultrasonic probe 20 coming out of the tube 13 of the ultrasonic probe holder 10 and pull the same upward.

FIGS. 6A and 6B are views illustrating states in which the ultrasonic probe 20 is housed in the ultrasonic probe holder 10. In FIGS. 6A and 6B, the same reference numerals are assigned to the same components as those in FIG. 4. Note that, in FIGS. 6A and 6B, the cable receiver 14 and the cable CB1 extending from the grip 22 of the ultrasonic probe 20 illustrated in FIG. 4 are not illustrated.

FIG. 6A illustrates a state before the ultrasonic probe 20 is housed in the ultrasonic probe holder 10. When the ultrasonic probe 20 is housed in the ultrasonic probe holder 10, this is inserted into the tube 13 of the ultrasonic probe holder 10 the head 21 first as illustrated in FIG. 6A.

FIG. 6B illustrates a state in which the ultrasonic probe 20 is housed in the ultrasonic probe holder 10. As illustrated in FIG. 6B, the ultrasonic probe 20 is housed in the ultrasonic probe holder 10 such that there is a gap between the output surface 23 and the bottom 12 of the ultrasonic probe holder 10 (a structure in which there is the gap between the output surface 23 and the bottom 12 is to be described later). That is, the output surface 23 of the ultrasonic probe 20 does not come into contact with the bottom 12 when the ultrasonic probe 20 is housed in the ultrasonic probe holder 10. As a result, breakage or the like of the output surface 23 of the ultrasonic probe 20 is prevented.

FIG. 7 is a perspective view of the ultrasonic probe holder 10, the holder support member 40, and the screw 50. In FIG. 7, the same reference numerals are assigned to the same components as those in FIG. 3. Note that, in FIG. 7, the cable receiver 14 of the ultrasonic probe holder 10 is also illustrated.

As illustrated in FIG. 7, the tube 13 of the ultrasonic probe holder 10 includes the protrusion 13a (refer to FIG. 3) and protrusions 13b, 13e, and 13f.

FIG. 8 is a view of the ultrasonic probe holder 10 as seen from above. In FIG. 8, the same reference numerals are assigned to the same components as those in FIG. 7. Note that, in FIG. 8, the holder support member 40 and the screw 50 illustrated in FIG. 7 are not illustrated.

As illustrated in FIG. 8, the tube 13 of the ultrasonic probe holder 10 includes protrusions 13a to 13h. The protrusions 13a to 13h in plate-shapes (for example, refer to the protrusion 13a in FIG. 3, and the protrusions 13a, 13b, 13e, and 13f in FIG. 7) extend in a vertical direction (z axis direction).

The protrusion 13a is provided on the inner side on the back side of the tube 13 and on a left side as seen from the front. The protrusion 13b is provided on the inner side on the front side of the tube 13 and on the left side as seen from the front. The protrusion 13c is provided on the inner side on the back side of the tube 13 and on a right side as seen from the front. The protrusion 13d is provided on the inner side on the front side of the tube 13 and on the right side as seen from the front.

The protrusions 13e and 13f are provided on the inner side on a lateral side of the tube 13 and on the left side as seen from the front. The protrusions 13g and 13h are provided on the inner side on the lateral side of the tube 13 and on the right side as seen from the front.

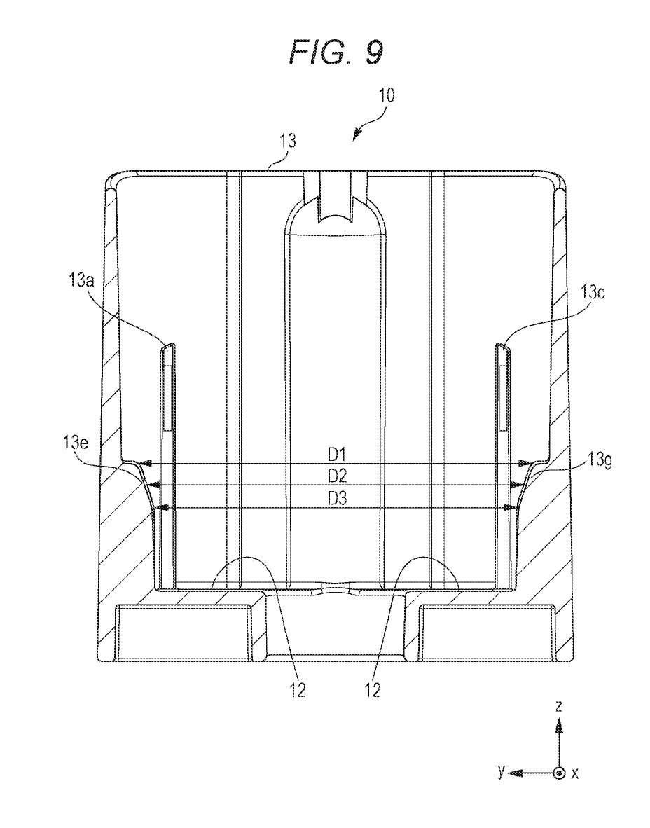

FIG. 9 is a cross-sectional view as seen in arrow AA direction of FIG. 8. In FIG. 9, the same reference numerals are assigned to the same components as those in FIG. 8.

As illustrated in FIG. 9, the protrusions 13e and 13g protrude in directions to be opposed to each other. A space between the protrusions 13e and 13g gradually narrows toward the bottom 12.

For example, D1 to D3 illustrated in FIG. 9 indicate the space between the protrusions 13e and 13g. D2 is smaller than D1 and D3 is smaller than D2.

The protrusions 13f and 13h illustrated in FIG. 8 are similar to the above-described protrusions 13e and 13g. That is, the space between the protrusions 13f and 13h gradually narrows toward the bottom 12.

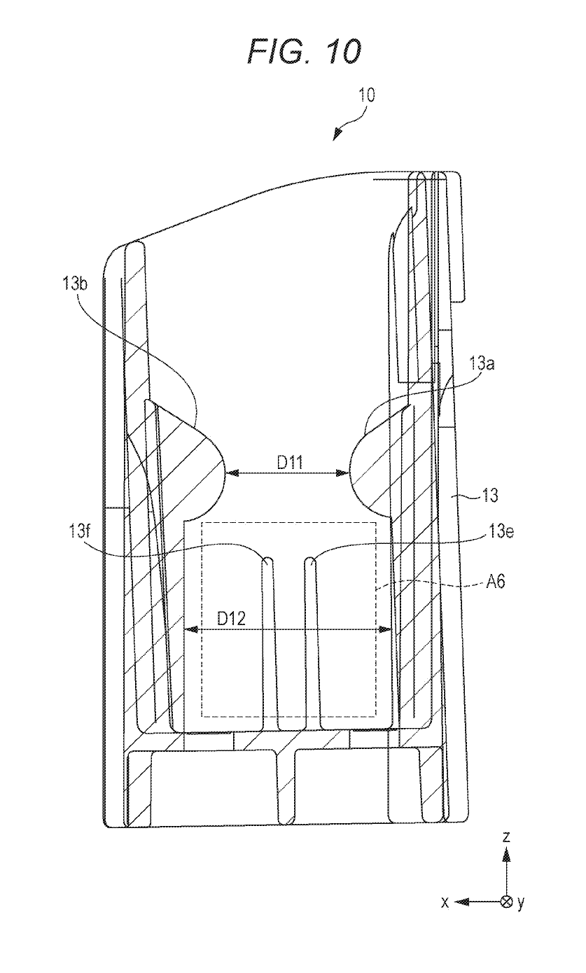

FIG. 10 is a cross-sectional view as seen in arrow BB direction of FIG. 8. In FIG. 10, the same reference numerals are assigned to the same components as those in FIG. 8.

As illustrated in FIG. 10, the protrusions 13a and 13b protrude in directions so as to be opposed to each other. The protrusions 13a and 13b are such that an upper portion protrudes than a lower portion. That is, a space between the protrusions 13a and 13b is narrower at the upper portion than at the lower portion. For example, D11 illustrated in FIG. 10 is smaller than D12.

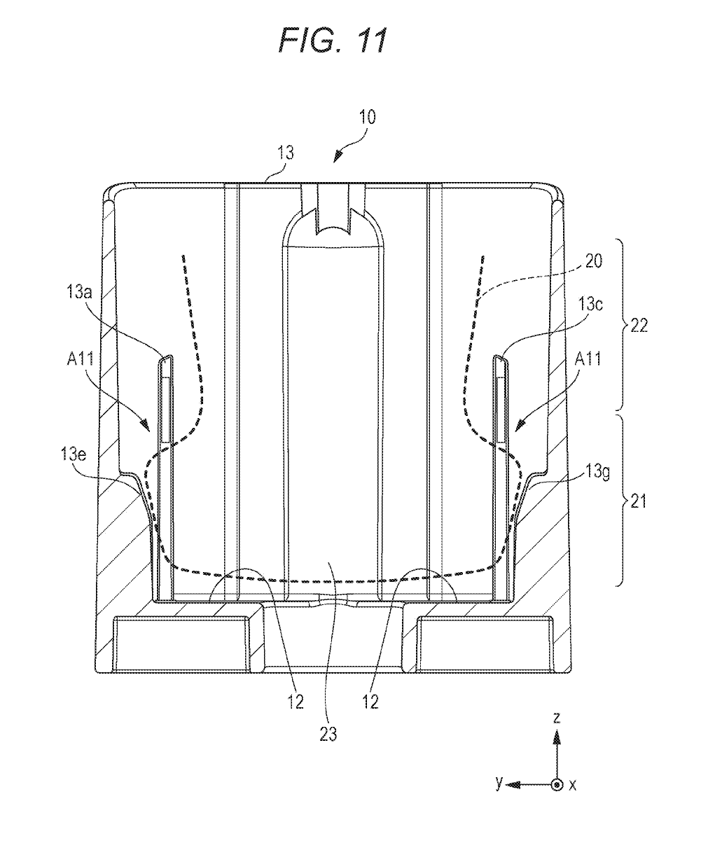

FIG. 11 is a view for illustrating a housed state of the ultrasonic probe 20. In FIG. 11, the same reference numerals are assigned to the same components as those in FIG. 9. Note that, dotted line in FIG. 11 indicates the head 21 and a part of the grip 22 of the ultrasonic probe 20 housed in the ultrasonic probe holder 10.

As illustrated in FIG. 11, a width (width in the y axis direction) of the head 21 of the ultrasonic probe 20 is narrowed toward the output surface 23. A side surface of the head 21 is brought into contact with the protrusions 13e and 13g the space therebetween gradually narrows toward the bottom 12 and the protrusions 13f and 13h (refer to FIG. 8) not illustrated in FIG. 11.

Movement of the ultrasonic probe 20 in the direction toward the bottom 12 (−z axis direction) is blocked by the contact between the side surface of the head 21 and the protrusions 13e to 13h. When the side surface of the head 21 and the protrusions 13e to 13h are in contact with each other, there is the gap between the output surface 23 of the ultrasonic probe 20 and the bottom 12. That is, the protrusions 13e to 13h support the head 21 of the ultrasonic probe 20 such that the output surface 23 of the ultrasonic probe 20 does not come into contact with the bottom 12. As a result, the output surface 23 of the ultrasonic probe 20 is protected from breakage and the like.

Also, a width (width in the y axis direction) of the output surface 23 is narrower than the space between the protrusions 13e and 13g and the space between the protrusions 13f and 13h. As a result, the ultrasonic probe 20 is housed in the ultrasonic probe holder 10 such that the output surface 23 does not come into contact with the protrusions 13e to 13h.

As illustrated in FIG. 10, the space between the protrusions 13a and 13b is narrower at the upper portion than at the lower portion. Also, the space between the protrusions 13c and 13d is narrower at the upper portion than at the lower portion. The space between the upper portion of the protrusion 13a and the upper portion of the protrusion 13b and the space between the upper portion of the protrusion 13c and the upper portion of the protrusion 13d are narrower than a thickness of the head 21 of the ultrasonic probe 20 (the thickness in the x axis direction).

The ultrasonic probe holder 10 is formed of a flexible resin or the like. Therefore, when the ultrasonic probe 20 is housed in the tube 13, the space between the upper portion of the protrusion 13a and the upper portion of the protrusion 13b and the space between the upper portion of the protrusion 13c and the upper portion of the protrusion 13d are widened by passage of the head 21. As a result, the head 21 of the ultrasonic probe 20 is housed in the lower portion of the protrusions 13a to 13d (space indicated by dotted line A6 in FIG. 10). When the head 21 of the ultrasonic probe 20 is housed in the lower portion of the protrusions 13a to 13d, the space of the protrusions 13a to 13d widened when the head 21 passes through returns to the original space due to flexibility of the ultrasonic probe holder 10.

As indicated by arrow A11 in FIG. 11, the width of the head 21 is narrowed toward the grip 22. A portion where the width of the head 21 narrows (portion indicated by arrow A11) comes into contact with the upper portions of the protrusions 13a to 13d. That is, the protrusions 13a to 13d lock the head 21 such that the ultrasonic probe 20 housed in the ultrasonic probe holder 10 does not come out of the ultrasonic probe holder 10.

In order to take out the ultrasonic probe 20 housed in the ultrasonic probe holder 10 from the ultrasonic probe holder 10, the ultrasonic probe 20 is pulled out in a direction opposite to the bottom 12 (for example, the +z axis direction in FIG. 11) by applying a predetermined force. For example, as described above, the ultrasonic probe holder 10 has flexibility. When the ultrasonic probe 20 is pulled out with a predetermined force, the space between the upper portion of the protrusion 13a and the upper portion of the protrusion 13b and the space between the upper portion of the protrusion 13c and the upper portion of the protrusion 13d widen by the passage of the head 21, so that the ultrasonic probe 20 may be taken out from the ultrasonic probe holder 10.

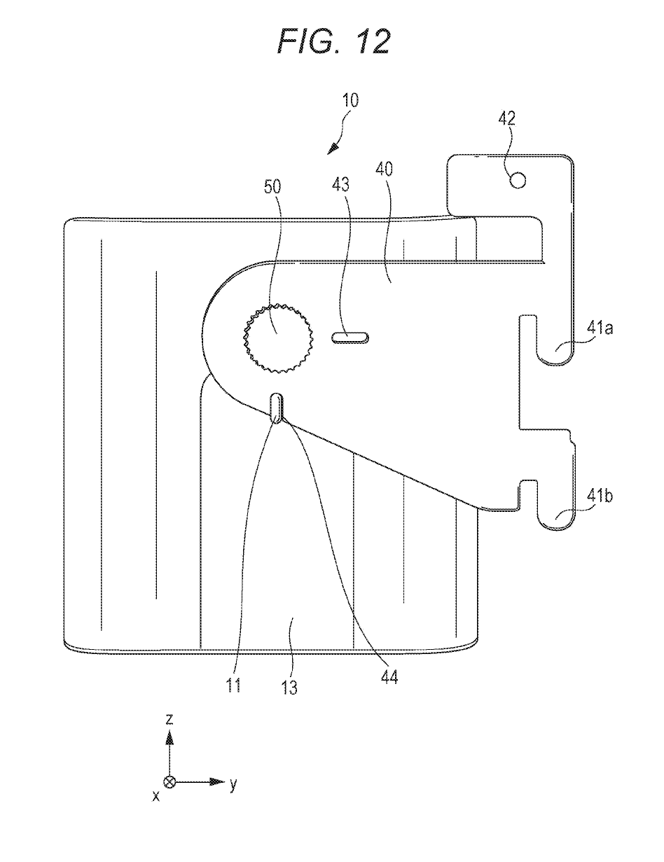

FIG. 12 is a view illustrating the ultrasonic probe holder 10 and the holder support member 40 when the ultrasonic probe holder 10 is attached to the ultrasonic diagnostic device 30. In FIG. 12, the same reference numerals are assigned to the same components as those in FIG. 2.

When attaching the ultrasonic probe holder 10 to the ultrasonic diagnostic device 30, as illustrated in FIG. 12, the holder support member 40 is fixed to the ultrasonic probe holder 10. For example, the cutout 44 of the holder support member 40 is fitted to the protrusion 11 provided on the back surface of the tube 13, and the screw 50 is tightened. Then, as illustrated in FIG. 2, the holder support member 40 to which the ultrasonic probe holder 10 is fixed to the back surface of the ultrasonic diagnostic device 30. As a result, the ultrasonic probe holder 10 is fixed to the ultrasonic diagnostic device 30.

Note that, the ultrasonic probe holder 10 may be fixed to the holder support member 40 after attaching the holder support member 40 to the ultrasonic diagnostic device 30.

FIG. 13 is a view illustrating the ultrasonic probe holder 10 and the holder support member 40 when the ultrasonic probe holder 10 is housed. In FIG. 13, the same reference numerals are assigned to the same components as those in FIG. 12. Note that, in FIG. 13, the screw 50 is not illustrated.

When the ultrasonic probe holder 10 is housed in the housing case, the housing bag or the like, the holder support member 40 is rotated as illustrated in FIG. 13 from the state in FIG. 12 (rotated clockwise by 90 degrees) to be fixed to the ultrasonic probe holder 10. For example, the hole 43 of the holder support member 40 is aligned with the protrusion 11 provided on the back surface of the tube 13, and the screw 50 is tightened.

As illustrated in FIGS. 12 and 13, the holder support member 40 is attached in different direction with respect to the tube 13. In a case of an attaching direction illustrated in FIG. 13, an outer edge of the holder support member 40 as seen from the back of the ultrasonic probe holder 10 is inside an outer edge of the ultrasonic probe holder 10. In other words, the holder support member 40 is hidden by the tube 13. As a result, a housing space of the ultrasonic probe holder 10 and the holder support member 40 in the housing case, the housing bag or the like may be reduced.

On the other hand, in a case of the attaching direction illustrated in FIG. 12, the outer edge of the holder support member 40 as seen from the back of the ultrasonic probe holder 10 protrudes from the outer edge of the ultrasonic probe holder 10. For example, a portion of the protrusions 41a and 41b and the hole 42 protrudes from the outer edge of the ultrasonic probe holder 10 (for example, in FIG. 12, the protrusions 41a and 41b and the hole 42 protrude in the +y axis direction). As illustrated in FIG. 2, the protrusions 41a and 41b protruding from the ultrasonic probe holder 10 are inserted into the holes 31a and 31b on the back surface of the ultrasonic diagnostic device 30, and the hole 42 is fitted to the protrusion 32.

When the ultrasonic probe holder 10 is housed, the plate-shaped holder support member 40 is attached to the ultrasonic probe holder 10 as illustrated in FIG. 13, so that this is prevented from being lost.

As described above, the ultrasonic probe holder 10 which holds and houses the ultrasonic probe 20 including the head 21 including the output surface 23 which outputs ultrasonic waves and the grip 22 extending from the head 21 in the direction opposite to the output surface 23 includes the bottom 12 including the opening A5 the size of which is such that the head 21 and the grip 22 of the ultrasonic probe 20 do not pass therethrough. Also, the ultrasonic probe holder 10 includes the tube 13 extending from the bottom 12, and including the opening A1 at an extended end having the size such that the head 21 and the grip 22 of the ultrasonic probe 20 may pass therethrough and the slit A2 connected to the opening A1 and the opening A5 of the bottom 12.

As a result, the ultrasonic probe holder 10 may house the ultrasonic probe 20 so as to protect the output surface 23 thereof as illustrated in FIG. 6B, for example, when the ultrasonic probe is not used, and may hold the ultrasonic probe 20 such that the gel does not drop as illustrated in FIG. 1, for example, when the ultrasonic probe 20 is used.

Note that, although the holder support member 40 is attached to the ultrasonic diagnostic device 30 above, there is no limitation. For example, the holder support member 40 may be detachably attached to a cart on which the ultrasonic diagnostic device is mounted.

The ultrasonic probe 20 may also be held by the ultrasonic probe holder 10 such that the head 21 faces downward. For example, when no gel is applied to the output surface 23, the ultrasonic probe 20 may be held by the ultrasonic probe holder 10 such that the head 21 faces downward.

Also, the number and shape of the protrusions 13a to 13h are not limited to the above-described examples.

For example, it is sufficient that the protrusions 13a to 13d may lock the head 21 of the ultrasonic probe 20 inserted from the opening A1 toward the bottom 12 such that the housed ultrasonic probe 20 does not come out of the ultrasonic probe holder 10. Also, the protrusions 13e to 13h only have to support the head 21 such that the output surface 23 of the ultrasonic probe 20 inserted from the opening A1 toward the bottom 12 does not come into contact with the bottom 12.

The above-described embodiment merely describes an example of substantiation when carrying out the present invention, and the technical scope of the present invention cannot be interpreted in a limited manner by the same. That is, the present invention may be variously carried out without departing from the gist or the main characteristics thereof.

Although embodiments of the present invention have been described and illustrated in detail, the disclosed embodiments are made for purposes of illustration and example only and not limitation. The scope of the present invention should be interpreted by terms of the appended claims.

What is claimed is:

1. An ultrasonic probe holder which holds and houses an ultrasonic probe including a head including an output surface which outputs ultrasonic waves and a grip extending from the head, the ultrasonic probe holder comprising:
a bottom that includes a first opening having a size such that the head and the grip of the ultrasonic probe do not pass therethrough; and
a tube that extends from the bottom and includes a second opening having a size such that the head and the grip of the ultrasonic probe pass therethrough at an extended end and a slit connected to the second opening and the first opening on the bottom.

2. The ultrasonic probe holder according to claim 1,
wherein the tube has a height such that a part of the grip comes out of the tube when the grip is inserted from the second opening toward the bottom.

3. The ultrasonic probe holder according to claim 1,
wherein the slit has a width such that a cable extending from the grip passes therethrough.

4. The ultrasonic probe holder according to claim 1,
wherein the tube includes a first protrusion on an inner side, and
the first protrusion supports the head such that the output surface of the ultrasonic probe inserted from the second opening toward the bottom does not come into contact with the bottom.

5. The ultrasonic probe holder according to claim 1,
wherein the tube includes a second protrusion on the inner side, and
the second protrusion locks the head of the ultrasonic probe inserted from the second opening toward the bottom such that the ultrasonic probe housed in the ultrasonic probe holder does not come out of the ultrasonic probe holder.

6. The ultrasonic probe holder according to claim 1,
wherein the tube has flexibility.

7. The ultrasonic probe holder according to claim 1,
wherein a cable receiver for hanging or winding a cable extending from the grip is provided on an outer periphery of the tube.

8. The ultrasonic probe holder according to claim 1, further comprising:
a holder support member that is detachably attached to an outer side of the tube,
wherein the holder support member is detachably attached to an ultrasonic diagnostic device.

9. The ultrasonic probe holder according to claim 1, further comprising:
a holder support member that is detachably attached to an outer side of the tube,
wherein the holder support member is detachably attached to a cart on which an ultrasonic diagnostic device is mounted.

10. The ultrasonic probe holder according to claim 8,
wherein the holder support member is attached while changing a direction with respect to the tube so as to be hidden by the tube.
